(19) United States
(12) Patent Application Publication
(10) Pub. No.: US 2024/0066193 A1
(43) Pub. Date: Feb. 29, 2024

(54) WEARABLE LEAK-PROOF BREAST SUCTION DEVICE

(71) Applicant: SHENZHENSHI LUTEJIACHENG SUPPLYCHAIN MANAGEMENT CO., LTD., SHENZHEN CITY (CN)

(72) Inventors: WANYUAN CHEN, SHENZHEN CITY (CN); QINYANG SU, SHENZHEN CITY (CN)

(21) Appl. No.: 18/093,350

(22) Filed: Jan. 5, 2023

(30) Foreign Application Priority Data
Aug. 27, 2022 (CN) 202222266427.9

(51) Int. Cl. A61M 1/06

(52) U.S. Cl. CPC A61M 1/064; A61M 1/067

(57) ABSTRACT

A wearable leak-proof breast suction device is provided. The breast suction device includes a breast suction device body. A breast suction cover is disposed on the breast suction device body. A sucker is disposed on a central bulging part of the breast suction cover, and a central depression part of the sucker has a through hole connected to a three-way member of the breast suction device body. The sucker covers a breast when the breast suction device body is in operation. A connecting pipe is disposed between the sucker and the breast suction cover, and is connected to a through hole and extends in a direction toward the three-way member. Each of the connecting pipe, the sucker and the breast suction cover is flexible. An inner edge of the connecting pipe is curved at a connection with an edge of the through hole of the sucker.

### CROSS-REFERENCE TO RELATED PATENT APPLICATION

This application claims the benefit of priority to Patent Application No. 202222266427.9, filed on Aug. 27, 2022 in People's Republic of China. The entire content of the above identified application is incorporated herein by reference.

### FIELD OF THE DISCLOSURE

The present disclosure relates to a baby care product, and more particularly to a wearable leak-proof breast suction device.

### BACKGROUND OF THE DISCLOSURE

Breastfeeding is beneficial and harmless to the healthy growth of babies. Breast milk, especially colostrum, is irreplaceable high-quality milk for any dairy products, contains rich nutrition that babies need. In addition to being beneficial to baby's health, breastfeeding can also prevent postpartum anemia, speed up the recovery of maternal health, and decrease the probability of developing ovarian cancer and breast cancer of breastfeeding mothers.

Many mothers have little or even no breast milk, and milk secretion needs to be stimulated through artificial massage or breast suction devices. However, air leakage in the conventional breast suction device often occurs during physical activities, resulting in poor sucking effect and frequent breast milk leakage.

### SUMMARY OF THE DISCLOSURE

In response to the above-referenced technical inadequacies, the present disclosure provides a wearable leak-proof breast suction device, which can effectively prevent a poor sucking effect and breast milk leakage caused by air leakage of a breast suction device.

In one aspect, the present disclosure provides a wearable leak-proof breast suction device, which includes a breast suction device body. A breast suction cover is disposed on the breast suction device body. The breast suction cover is configured to cover a breast when the breast suction device body is in operation. A sucker is disposed on a central bulging part of the breast suction cover, a central depression part of the sucker has a through hole connected to a three-way member of the breast suction device body and the sucker is configured to cover breast when the breast suction device body is in operation.

In certain embodiments, a connecting pipe is disposed between the sucker and the breast suction cover, and the connecting pipe is connected to the through hole and extends in a direction toward the three-way member.

In certain embodiments, each of the connecting pipe, the sucker, and the breast suction cover is made of a flexible material.

In certain embodiments, an inner edge of the connecting pipe is curved at a connection with an edge of the through hole of the sucker.

In certain embodiments, a surface area of a contact area of the sucker is less than a surface area of the breast suction cover.

In certain embodiments, the breast suction cover has a curved edge.

In certain embodiments, the connecting pipe, the sucker, and the breast suction cover are integrally formed.

In certain embodiments, each of the connecting pipe, the sucker, and the breast suction cover is made of a silicone material.

Adopting the above technical contents, the beneficial effects of the present disclosure are as follows:

1. In the wearable leak-proof breast suction device provided by the present disclosure, the sucker is disposed on the central bulging part of the breast suction cover, and the breast suction cover is configured to cover the breast when the breast suction device body is in operation. The central part of the sucker is recessed in the direction toward the three-way member of the breast suction device, and the sucker has the through hole, which is connected to the three-way member, thereby breast milk sucked out by the breast suction device body can flow into the three-way member through the through hole. The sucker clings to the breast for the nipple to be placed in the through hole when the breast suction device body is in operation. With a cooperation between the breast suction cover and the sucker, airtightness of the breast suction device body during operation is further improved, and the air leakage and the milk leakage can be effectively prevented.

2. The connecting pipe is disposed between the sucker and the breast suction cover, and one end of the connecting pipe is connected to the through hole, and another end of the connecting pipe is connected to the three-way member. Each of the connecting pipe, the sucker, and the breast suction cover is flexible. The connecting pipe can be used for the sucker to follow the movement of the breasts, so that the sucker can fit the breasts better, thereby preventing the air leakage.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic structural view of a wearable leak-proof breast suction device of the present disclosure;

FIG. 2 is a side view corresponding to FIG. 1;

FIG. 3 is a schematic structural view of a sucker of the wearable leak-proof breast suction device of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

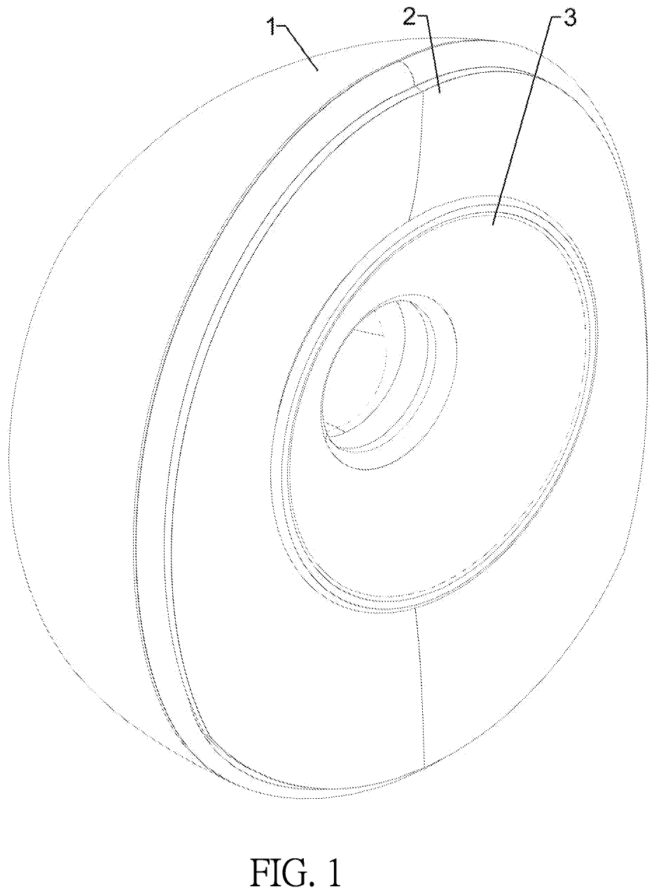

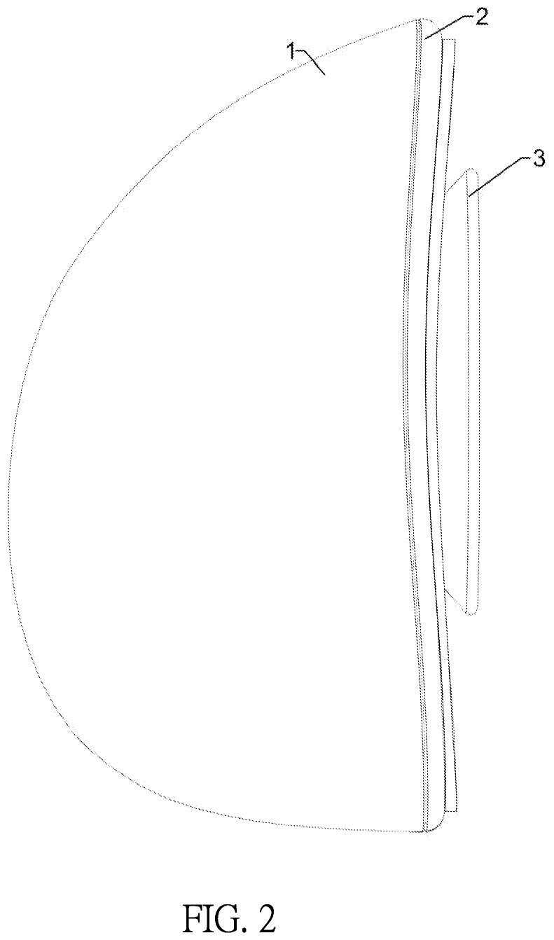

Referring to FIG. 1 and FIG. 2, the present disclosure provides a wearable leak-proof breast suction device. A sucker 3 is disposed on a central bulging part of a breast suction cover 2 on a breast suction device body 1, and the breast suction cover 2 is configured to cover a breast when the breast suction device body 1 is in operation. A central part of the sucker 3 is recessed in a direction toward a three-way member of the breast suction device. The sucker 3 has a through hole 311, which is connected to the three-way member, so that breast milk sucked out by the breast suction device body 1 can flow into the three-way member through the through hole 311. The sucker 3 clings to the breast for a nipple to be placed in the through hole 311 when the breast suction device body 1 is in operation. With a cooperation between the breast suction cover 2 and the sucker 3, airtightness of the breast suction device body 1 during operation is further improved, and air leakage and milk leakage are effectively prevented.

It should be noted that, the sucker 3 has a trumpet part 33, in which a small mouth of the trumpet part 33 is the through hole 311, a big mouth of the trumpet part 33 is a breast insertion part, and an oblique radian of the trumpet part 33 adapts to a concave-convex radian of the breast. In another embodiment, the through hole 311 is not arranged in the central part of the sucker 3, but the through hole 311 is still spatially communicated with the three-way member, and such type of sucker 3 can be adapted to breasts of different sizes.

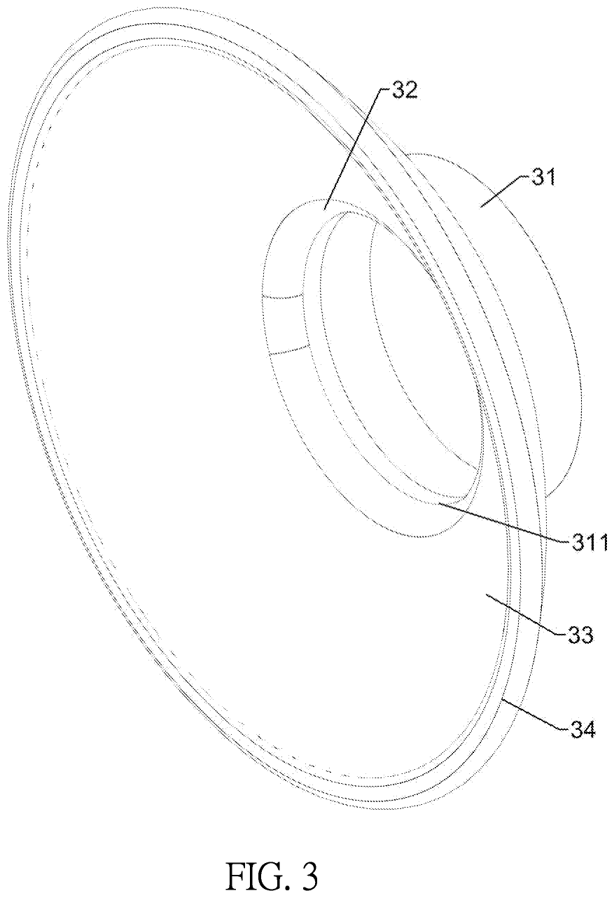

As shown in FIG. 3, a connecting pipe 31 is disposed between the sucker 3 and the breast suction cover 2. One end of the connecting pipe 31 is connected to the through hole 311, and another end of the connecting pipe 31 is connected to the three-way member. Each of the connecting pipe 31, the sucker 3, and the breast suction cover 2 is made of a flexible material. The connecting pipe 31 can be used for the sucker 3 to follow a movement of the breasts, so that the sucker 3 can fit the breasts better, thereby preventing the air leakage.

It should be noted that a transition part 32 is disposed on an inner edge of the connecting pipe 31 and an edge of the through hole 311 of the sucker 3, and the transition part 32 is arranged in a curved shape to connect the connecting pipe 31 and the through hole 311 of the sucker 3. The transition part 32 can improve a comfort level when putting the breast into the through hole 311, thereby improving an overall user experience.

It should be noted that each of the connecting pipe 31, the sucker 3, and the breast suction cover 2 is made of a flexible material, the flexible sucker 3 can improve a comfort level when contacting with the skin around the breasts, and the flexible connecting pipe 31 can produce deformation that does not affect the effect of breast milk sucking when the breast suction device moves or the human body moves, so as to keep the sucker 3 covering the breast. Each of the connecting pipe 31, the suction cup 3 and the breast shield 2 is preferably made of a silicone material.

In one particular embodiment, a surface area of a contact surface of the sucker 3 is less than a surface area of the breast suction cover 2. When the breast suction device moves or the human body moves, the sucker 3 with a smaller size provides a greater flexibility. The breast suction cover 2 with a larger size can clad the breast in a larger area to improve the overall airtightness of the breast suction device during operation.

It should be noted that an edge of the breast suction cover 2 is a curved edge 34, and the curved edge 34 can effectively avoid the breasts from being cut or scratch by a sharp edge.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

What is claimed is:

1. A wearable leak-proof breast suction device, comprising:
a breast suction device body,
wherein a breast suction cover is disposed on the breast suction device body, and the breast suction cover is configured to cover a breast when the breast suction device body is in operation;
wherein a sucker is disposed on a central bulging part of the breast suction cover, a central depression part of the sucker has a through hole connected to a three-way member of the breast suction device body, and the sucker is configured to cover the breast when the breast suction device body is in operation.

2. The wearable leak-proof breast suction device according to claim 1, wherein a connecting pipe is disposed between the sucker and the breast suction cover, and the connecting pipe is connected to the through hole and extends in a direction toward the three-way member.

3. The wearable leak-proof breast suction device according to claim 2, wherein each of the connecting pipe, the sucker, and the breast suction cover is made of a flexible material.

4. The wearable leak-proof breast suction device according to claim 2, wherein an inner edge of the connecting pipe is curved at a connection with an edge of the through hole of the sucker.

5. The wearable leak-proof breast suction device according to claim 1, wherein a surface area of a contact area of the sucker is less than a surface area of the breast suction cover.

6. The wearable leak-proof breast suction device according to claim 1, wherein the breast suction cover has a curved edge.

7. The wearable leak-proof breast suction device according to claim 2, wherein the connecting pipe, the sucker, and the breast suction cover are integrally formed.

8. The wearable leak-proof breast suction device according to claim 3, wherein each of the connecting pipe, the sucker, and the breast suction cover is made of a silicone material.
